(19) United States
(12) Patent Application Publication
(10) Pub. No.: US 2007/0224248 A1
(43) Pub. Date: Sep. 27, 2007

(54) METHOD AND COMPOSITIONS FOR TREATING ACNE

(76) Inventor: Steven A. Smith, Tulsa, OK (US)

Correspondence Address: Miles Smith, P.O. Box 702418, Tulsa, OK, 74170, US

(21) Appl. No.: 11/277,083

(22) Filed: Mar. 21, 2006

(51) Int. Cl. A61K 33/34; A61K 33/26; A61K 33/32; A61K 33/00

(52) U.S. Cl. 424/440; 424/642; 424/646; 424/705; 424/723; 424/451; 424/464; 424/635

(57) ABSTRACT

A method and composition for treating acne. The present invention involves treatment of this medical condition with an oral administration of a mixture comprised of inorganic nickel compound(s) such as nickel sulfate, inorganic bromide compound(s) such as potassium bromide, inorganic zinc compound(s), and sulphur.

### REFERENCES CITED

### U.S. PATENT DOCUMENTS

U.S. Pat. No. 5,171,581 December 1992 Smith et al.

U.S. Pat. No. 5,681,593 October 1997 Smith et al.

U.S. Pat. No. 6,613,800 B1 September 2003 Smith et al.

### OTHER PUBLICATIONS

Kolipinski, L., M.D., “On the Uses of Nickel Sulphate in Medicine”, Cyclopaedia & Medical Bulletin, pp. 348-355, (1911).
Weingartner et al. “Composition of the First . . . in Concentrated Aqueous NiCl₂ and NiBr₂ solutions”. J. Chem. Soc., Faraday Trans. 1, 75 (12), 2700-11.
Gawkrodger, et al., “Nickel Dermatitis: The Reaction to Oral Nickel Challenge”, Brit. J. Dermat., 115, pp. 33-38 (1986).
Sjovall, et al., “Oral Hyposensitization in Nickel Allergy”, J. Amer. Acad. Dermatitis, V. 17, No. 5, Part 1, November 1987.
DaCosta, J. M., “Observations on the Salts of Nickel, Especially the Bromide of Nickel”, The Medical News, vol. XLIII, No. 13 (1883).

### BACKGROUND OF THE INVENTION

The skin is the largest organ of the human body. It serves many purposes but its primary functions are to serve as a barrier to the environment, protect against water loss, and regulate body temperature. The skin is a complex, fibrous, elastic structure encasing all the living tissues and organs of the body. The outermost layer of the skin, the epidermis, is continuous with the mucous membrane epithelium of the respiratory, digestive, and urogenital systems. It overlies the dermis (corium), a connective tissue layer of mesodermal origin. The third and deepest layer of the skin is composed of subcutaneous fat.

Acne is the single most common reason patients between the ages of 15 to 45 years consult a skin doctor. Acne is a skin disorder caused by the inflammation and/or infection of hair follicles with or without sebaceous gland over activity. The majority of acne cases are pleomorphic, ranging in the types of lesions visible: comedones, papules, pustules, nodules, and cysts. All acne types can be subdivided into one of two classes: noninflammatory and inflammatory acne. Noninflammatory acne is characterized by open and closed comedones, composed of a compact collection of keratin, sebum, and bacteria, which causes hair follicle ducts to dilate. Inflammatory acne is distinguished by inflammation of the skin, presence of papules, pustules, and nodulocysyic lesions which have a tendency to scar. Acne usually materializes on the face; however it can also occur on the chest, back, neck, and upper arms. Although acne is usually associated with puberty, mild degrees of acne are often observed at birth. Furthermore, more than half of all adult women and a quarter of adult men are diagnosed with some form of adult acne.

There are many different variations of acne and they are usually classified according to their predominant lesion morphology. Acne vulgaris is the most common variation of acne diagnosed. Symptoms of acne vulgaris usually consist of comedones, cysts, papules, and pustules; however not all are required to be present for diagnosis. Acne vulgaris is a chronic condition that may persist for several years.

Another relatively common type of acne is rosacea or “middle-age acne.” Acne rosacea is a chronic inflammatory disorder affecting the blood vessels and pilosabaceous units of the face and occasionally the neck and upper trunk. Acne rosacea is characterized by papules and pustules superimposed upon a background of erythema and telangiectasia. It can be distinguished from acne vulgaris by the absence of comedones, the presence of erythema and telangiectasia, the gradual onset in middle life, and localized distribution within the central third of the face.

The exact cause of acne is not known, however it is believed that there are three main factors involved in acne's pathogenesis: androgens, bacteria, and free fatty acids. Androgens are intrinsic hormones known to stimulate sebaceous glands causing them to enlarge and increase their sebum production promoting comedone formation. The bacterium, Propionibacterium acnes (P. acnes) has been shown to produce follicular lipases, proteases, and hyaluronidases. These enzymes are believed to play an important role in inflammation. In some cases P. aureus causes an infection within the hair follicle, known as gram negative folliculitis, leaving scars. Lastly, P. acnes is also known to breakdown the sebum in a follicle into free fatty acids which also cause inflammation.

Most medications are aimed at controlling acne rather than eradicating it. There are three main categories of medication for acne treatment: topical comedolytic agents, systemic antibiotics, and systemic retinoids. Frequently prescribed topical comedolytic agents include retinoic acid (Retin A), benzoyl peroxide, azelaic acid, alpha-hydroxy acid, and salicylic acid. However, these medications can cause dryness, redness, peeling, skin irritation, and can cause a patient's condition to worsen before improving.

Systemic antibiotics are primarily used for the treatment of inflammatory acne. They include tetracycline, doxycycline, minocycline, erythromycin, and ampicillin. Some side effects of these antibiotics consist of nausea, upset stomach, diarrhea, yeast infections, sun sensitivity, headaches, dizziness, and bacterial resistance. Isotretinoin, commonly referred to as Accutane®, is the most commonly used systemic retinoid. However it is reserved for severe resistant cases only, due to its teratogenic properties. Isotretinoin side effects can also include chapped lips, dry skin, elevated liver function tests, and elevated plasma lipids.

Despite the forgoing advances, there is a need in the art for new effective treatments for acne which do not produce the side effects associated with traditional acne therapies.

### SUMMARY OF THE INVENTION

The present invention involves treatment of all types of acne with an oral administration of a mixture of four primary active ingredients: inorganic bromide compound(s), inorganic nickel compound(s), inorganic zinc compound(s), and sulphur. In one preferred embodiment of the invention, a quantity of copper is included to avoid the copper deficiency that might otherwise occur due to the zinc supplementation.

The exact mechanism of action of this invention is not known, however the combination of these ingredients has been very successful in treating a wide range of inflammatory and noninflammatory acne.

Bromide enhances phagocytic and bactericidal activity of neutrophils. This interferes with the pathogenic step of corynebacteriom acnes and other bacteria (gram negative and gram positive) that are involved in the cascade of events that result in acne lesions; Steele, R. W. and Woody, R. C.; Enhanced Neutrophil Function in Children on Bromide Thaerapy; The American Journal of the Medical Sciences; 302:145-147; (1991). Other possible mechanisms of action include a direct effect on sebaceous glands that helps normalize the texture of the skin.

Nickel ions are a standard repellant for bacterial chemotaxis; Bray, D, Bourret, R. B., and Simon, M. I.; Computer Simulation of the Phosphorylation Cascade Controlling Bacterial Chemotaxis; The American Society for Cell Biology; 4:469-481; (1993). This interferes with the bacteria's ability to migrate into the skin and pilosebaceous unit, thus interfering with the bacterial induced step of acne pathogenesis. Other possible mechanisms include antioxidant and other anti-inflammatory properties of nickel ions.

Zinc is an antioxidant nutrient that stimulates the activity of enzymes which promote biochemical reactions in the human body. Among its many functions, zinc helps maintain a healthy immune system, is needed for wound healing, helps maintain your sense of taste and smell, and is needed for DNA synthesis; The Benefits of Zinc. Dr. George Obikoya. 2004. The Vitamins & Nutrition Center. 19 Jul. 2005 <http://www.vitamins-nutrition.org/vitamins/zinc.html>.

Organic sulphur is a naturally occurring substance in the body and is radically different from sulfa drugs (sulfonamide antibiotics). Sulphur exhibits anti-bacterial, anti-parasitic, fungicidal, and keratolytic properties; Remington: The Science and Practice of Pharmacy. 19^{th} Edition, Mack Publishing Company, 1995.

### DETAILED DESCRIPTION AND EXAMPLES

The following examples illustrate various applications of the present invention. These cases are not to be construed to limit the claims in any manner whatsoever.

Clinical Studies in Treating Acne with Nickel, Bromide, and Zinc

Clinical case studies were performed on subjects selected from the patient base of Dr. Steven A. Smith M.D's clinic in Tulsa, Okla. (5801 E. 41^{st} St. Ste. 220). Dr. Smith was the sole study investigator for these cases. Dr. Smith received his BA in 1975 from Indiana Wesleyan University and his MD in 1979 from Indiana University School of Medicine. He completed his Internal Medicine Internship and Dermatology Residency at the University of Texas Health Science Center. Dr. Steven Smith is certified by the American Board of Dermatology and the American Board of Internal Medicine. He currently serves as a Clinical Associate Professor of Dermatology at the University of Oklahoma College of Medicine in Tulsa, Okla.

Subjects were selected on the basis of a history with acne. The subjects suffered from a wide range of acne variations: inflammatory acne, cystic scarring acne, adult onset acne, rosacea, peri-oral dermatitis and acne vulgaris. Evaluation criteria used were: types of lesions present, the quantity of lesions, and the location and recurrent nature of acne outbreaks. These evaluations were dependent on the history and examination of patients with these disease conditions.

Standard treatment charts were maintained on each subject, including a history of the patient's acne and its treatment pattern. Subjects were typically seen for evaluation, examination, and for adjustments to their medications on a monthly basis.

Each tablet of the study medication, Pharmaceutical Formulation U (PF U), contains 1.5 mg of nickel ions by the way of Nickel Sulphate; 15 mg of bromide ions from Potassium Bromide and Zinc Bromide; and 1.22 mcg of zinc ions by the way of Zinc Bromide. Three different dosages of PF U were used in evaluating the use of the minerals (nickel, bromide, and zinc) in low dosages to treat different variations of acne: 2 tablets, 3 tablets, and 4 tablets.

Case No. 1

This patient (hereafter A.B) is a 21 year old female weighing 150 lbs (68 kg) with a 13 year history of acne. AB was first evaluated on Mar. 16, 2005 for treatment of facial acne with occasional outbreaks on the back. Upon examination A.B. had a few dark macules on her upper central back, several large red papules on her left temple, a few pustules on her left cheek, two inflamed nodules on her right lateral chin, a cyst on the right temple, several comedones and one large red inflamed nodule on the right cheek. She was diagnosed with inflammatory acne. AB reported she had never received treatment and that OTC washes irritated her skin. She was promptly started on 3 tablets of PF U a day and Bactrin DS once a day. On Apr. 15, 2005, AB stated that her acne seemed to be clearing up, when examined it was apparent that her acne was improving. A few comedones were present on her forehead, a fading red papule on the right cheek, one cystic papule on the left lateral cheek, and one red papule on the upper central back. AB's dosage of PF U was increased to 4 tablets a day and she was started on Nicomide. On Jun. 17, 2005, A.B. asserted that her acne was doing significantly better. Her examination supported the fact that her facial acne had improved greatly. Several small papules were present on her forehead as well as a few red papules on her right cheek.

Case No. 2

This patient (hereafter SL) is a 31 year old female weighing 168 lbs (76 kg) with a history of acne. SL was first seen on Nov. 4, 2002 when she presented with numerous red papules and a few cystic lesions on her face. She was diagnosed with cystic acne. On Apr. 29, 2005, SL complained that her acne had worsened and upon examination two large red papules were present on her left lateral cheek, a large papule on her upper lateral forehead, and three medium sized papules on her right temple. At this time SL was instructed to take 2 tablets of PF U a day for two weeks and then increase her dosage to 3 tablets a day. SL was also started on one Bactrin DS a day. On Jun. 1, 2005, SL reported her acne had improved. Physical examination only showed a large papule on her central forehead, a cystic papule, and a few resolving papules on the temple forehead areas. SL's dose of PF U was reduced to two tablets a day. The following month, July 6, SL stated that her acne was responding very well to the treatment. Only one papule was present on the right cheek, three fading resolving papules on the left cheek, and one papule on the left lateral forehead.

Case No. 3

This patient (hereafter LD) is a 31 year old female with a long history of acne. LD was first evaluated on May 15, 1996 for the treatment of acne. At the time, LD had numerous plugged pores and papules on her back, a few red papules on her chest, several papules on her cheeks, and a pustule on her chin. She was diagnosed with adult onset acne. On Apr. 6, 2005 LD complained that her acne outbreaks were occurring more frequently. She presented with several small papules along her jawline, several larger papules on the left angle of the jaw, small papules on her chin, a few medium sized papules on the right medial cheek, several papules on the forehead, five red papules on the upper back, and one cystic papule on the right side of her neck. LD was started on two tablets of PF U a day, 100 mg of minocycline, and instructed to apply Benzyl Myosin Gel (BMG) twice a day. On her follow up visit, a month later (May 4, 2005), LD reported that her acne was doing better. She only exhibited fading red papules and one crusted papule on her forehead; one larger papule and a cystic papule on the chin; and one papule on the jawline. At this time LD's dosage was increased to 3 tablets of PF U a day. On Jul. 6, 2005 LD stated that her acne had improved significantly since the commencement of the treatment. A close examination only found one visible papule on the right cheek.

Case No. 4

This patient (hereafter AG) is a 14 year old male weighing 165 lbs (74 kg) with a short history of acne. AG was first seen on Jan. 25, 2005, he was seeking treatment for the numerous red papules and deep nodules located on his cheeks and a few resolving papules on his forehead and angle of the jaw. He was diagnosed with cystic scarring acne. AG was started on 2 tablets of PF U a day and 100 mg of minocycline a day. A month later, Feb. 25, 2005, AG communicated that his acne had slightly improved throughout the month. A physical examination showed that he had several papules and a few pustules on his cheeks and some faded red papules on his upper back; however no deep papules were present. This was a definite improvement. At this time his dosage was increased to 3 tablets of PF U daily. On AG's third visit (Mar. 22, 2005), he stated that his acne was doing much better, with the exception of an occasional flare. However, he did add that these flares did not last as long as before. His examination yielded the following results: forehead was clear, temples had fading redness, left cheek had four papules, right cheek had 3 papules, chin had three papules, and his upper back was free of active lesions and just contained fading papules. AG was instructed to continue taking 3 tablets of PF U every day and to return for follow up examination within two months. In May of 2005 AG reported that his skin had improved dramatically and his examination only yielded a few small comedones on his forehead, 3-4 papules on the left cheek, and one papule on the right cheek.

Case No. 5

This patient (hereafter MG) is a 32 year old female with no history of acne. However on her first evaluation, on Jan. 25, 2005, MG had many small to medium sized papules on her chin in addition to a few crusted papules. She was diagnosed with peri-oral dermatitis and acne vulgaris. On Feb. 22, 2005 MG complained about a bad flair she believed was brought on by her initial treatment. An examination found a few small comedones on her forehead, numerous closed comedones on her chin, and a crusted papule on the left medial cheek. She was started on 2 tablets of PF U a day and was kept on 50 mg of minocyclin a day. MG had her follow up visit on April 12, at which point she felt her acne had worsened. Her dosage was double to 4 tablets of PF U and the 50 mg of minocycline were replaced with 500 mg of amoxicillin a day. A month after the alterations to her treatment had been implemented (May 10), MG stated that her acne had improved. Upon examination a small crusted papule was noticeable on her chin and some tiny fading red papules were on her cheeks, nose, and forehead. MG was instructed to continue taking 4 tablets of PF U daily and to return in two months for a check up. On Jul. 12, 2005 the patient reported that her acne was doing really well and very rarely had breakouts.

The examples provided above are not meant to be exclusive. Many other variations of the present invention would be obvious to those skilled in the art, and are contemplated to be within the scope of the appended claims, including the full range of equivalency to which each element thereof is entitled.

Also, although the invention has been described as orally administered, the invention is not so limited and may be administered topically or by any other suitable means of administration.

What is claimed is:

1. The pharmaceutical formulation for the treatment of acne in a human patient comprising administering from about 0.0001-100 mcg of nickel/kg of body weight/day to promote the anti-acne effect of nickel ions.

2. The pharmaceutical formulation for the treatment of acne in a human patient comprising administering from about 0.0001-100 mcg of nickel/kg of body weight/day and an effective amount of bromide ions to promote the anti-acne effect of nickel ions.

3. The method of claim 1, wherein said nickel ions are derived from a non-toxic pharmaceutically acceptable nickel salt(s) or nickel compound(s).

4. The method of claim 2, wherein said nickel ions are derived from a non-toxic pharmaceutically acceptable nickel salt(s) or nickel compound(s).

5. The method of claim 1, wherein said nickel ions are derived from a non-toxic pharmaceutically acceptable nickel salt(s) or nickel compound(s) selected from a group such as NiSO₄, NiBr₂, NiCl₂, Nickel Acetate or any mixtures thereof, or similar compounds.

6. The method of claim 2, wherein said nickel ions are derived from a non-toxic pharmaceutically acceptable nickel salt(s) or nickel compound(s) selected from a group such as NiSO₄, NiBr₂, NiCl₂, Nickel Acetate or any mixtures thereof, or similar compounds.

7. The method of claim 2, wherein the amount of said bromide ions is between 0.1-5 mg of bromide/kg of body weight/day.

8. The method of claim 7, wherein said bromide ions are derived from a non-toxic pharmaceutically acceptable bromide salt(s) or bromide compound(s) selected from a group consisting of KBr, NaBr, NiBr₂, NH₄Br, and mixtures thereof, and similar compounds.

9. The method of claim 1, further comprising zinc ions.

10. The method of claim 2, further comprising zinc ions.

11. The method of claim 9, wherein the amount of said zinc ions is between 0.1-0.5 mg of zinc/kg of body weight/day.

12. The method of claim 10, wherein the amount of said zinc ions is between 0.1-0.5 mg of zinc/kg of body weight/day.

13. The pharmaceutical formulation of claim 11, wherein said zinc ions are derived from a non-toxic pharmaceutically acceptable zinc compound selected from a group consisting of zinc sulfate, zinc picolinate, zinc citrate, zinc acetate, zinc glycerate, zinc chloride, zinc gluconate, zinc monomethionine, and mixtures thereof, and similar compounds.

14. The pharmaceutical formulation of claim 12, wherein said zinc ions are derived from a non-toxic pharmaceutically acceptable zinc compound selected from a group consisting of zinc sulfate, zinc picolinate, zinc citrate, zinc acetate, zinc glycerate, zinc chloride, zinc gluconate, zinc monomethionine, and mixtures thereof, and similar compounds.

15. The method of claim 1, further comprising sulphur.

16. The method of claim 2, further comprising sulphur.

17. The method of claim 15, wherein the amount of said sulphur is between 0.0001 mcg-500 mcg of sulphur/kg of body weight/day.

18. The method of claim 16, wherein the amount of said sulphur is between 0.0001 mcg-500 mcg of sulphur/kg of body weight/day.

19. The pharmaceutical formulation of claim 17, wherein said sulphur is derived from a stable non-toxic pharmaceutically acceptable sulphur compound.

20. The pharmaceutical formulation of claim 18, wherein said sulphur is derived from a stable non-toxic pharmaceutically acceptable sulphur compound.

21. A pharmaceutical formulation of claim 1, further comprising Copper (II) Oxide.

22. A pharmaceutical formulation of claim 2, further comprising Copper (II) Oxide.

23. A method of treating acne in a human patient comprising simultaneously administering synergistic effective amounts between 0.001-5000 mcg of nickel/day, 1-1000 mg of bromide/day, 1-50 mg of zinc/day, and 0.00001-50 mg of sulphur/day.

24. A method of claim 23, wherein said formulation is in a dosage form selected from a group consisting of liquids, powders, capsules, caplets, pills, tablets, gelcaps, geltabs and lozenges.

25. A method of claim 23, further comprising administering to said patient an active agent selected from the group consisting topical antibacterial agents, topical retinoid, antibiotics, and combinations thereof.

26. A unit dose of the pharmaceutical formulation of claim 23 comprising from about 150 mg-1105 mg of aggregate said formulation.
